(19) United States
(12) Patent Application Publication
(10) Pub. No.: US 2022/0233091 A1
(43) Pub. Date: Jul. 28, 2022

(54) MEDICAL DEVICES CONFIGURED WITH NEEDLE ELECTRODES

(71) Applicant: NOVASCAN, INC., Milwaukee, WI (US)

(72) Inventors: Les BOGDANOWICZ, Park Ridge, IL (US); Terry DAGLOW, Allen, TX (US); Alexander GRYCUK, Mt. Prospect, IL (US); Paul Richard VOITH, Cedarburg, WI (US)

(21) Appl. No.: 17/695,748

(22) Filed: Mar. 15, 2022

Related U.S. Application Data
(63) Continuation-in-part of application No. 17/397,896, filed on Aug. 9, 2021.
Application No. 17/397,896 is a continuation-in-part of application No. 17/412,973, filed on Aug. 26, 2021.
(60) Provisional application No. 63/142,242, filed on Jan. 27, 2021.
(60) Provisional application No. 63/142,247, filed on Jan. 27, 2021.
(60) Provisional application No. 63/142,254, filed on Jan. 27, 2021.
(60) Provisional application No. 63/142,260, filed on Jan. 27, 2021.
(60) Provisional application No. 63/142,242, filed on Jan. 27, 2021.
(60) Provisional application No. 63/142,247, filed on Jan. 27, 2021.
(60) Provisional application No. 63/142,254, filed on Jan. 27, 2021.
(60) Provisional application No. 63/142,260, filed on Jan. 27, 2021.

(51) Int. Cl. A61B 5/0538; A61B 18/14

(52) U.S. Cl. CPC A61B 5/0538; A61B 2018/00875; A61B 18/1492; A61B 18/1477

(57) ABSTRACT

In various embodiments, a medical device includes an instrument head that includes a needle and two or more electrodes disposed on the needle; an impedance bridge coupled to the two or more electrodes; and a processor coupled to the impedance bridge. In various embodiments, a method for determining one or more tissue types present at a location associated with a needle of a medical device comprises recording, at one or more frequencies, one or more impedance measurements, wherein each impedance measurement is associated with two or more electrodes disposed on the needle of an instrument head of the medical device; comparing the one or more impedance measurements to one or more characteristic impedances associated with one or more tissue types; and determining, based on the one or more impedance measurements and the one or more characteristic impedances, one or more tissue types at the location associated with the needle.

### CROSS-REFERENCES TO RELATED APPLICATIONS

The present application is a continuation-in-part of the U.S. patent application titled, “IMPEDANCE-CALIBRATED DIAGNOSTIC MEDICAL DEVICES,” filed on Aug. 9, 2021, and having Ser. No. 17/397,896, which claims the benefit of U.S. Provisional Patent Application No. 63/142,242, filed Jan. 27, 2021; U.S. Provisional Patent Application No. 63/142,247, filed Jan. 27, 2021; U.S. Provisional Patent Application No. 63/142,254, filed Jan. 27, 2021; and U.S. Provisional Patent Application No. 63/142,260, filed Jan. 27, 2021. The present application is also a continuation-in-part of the U.S. patent application titled, “TECHNIQUES FOR CONTROLLING MEDICAL DEVICE TOOLS,” filed on Aug. 26, 2021, and having Ser. No. 17/412,973, which claims the benefit of U.S. Provisional Patent Application No. 63/142,242, filed Jan. 27, 2021; U.S. Provisional Patent Application No. 63/142,247, filed Jan. 27, 2021; U.S. Provisional Patent Application No. 63/142,254, filed Jan. 27, 2021; and U.S. Provisional Patent Application No. 63/142,260, filed Jan. 27, 2021. The subject matter of these related applications is hereby incorporated herein by reference.

### BACKGROUND

### Field of the Various Embodiments

Embodiments of the present disclosure relate generally to electronics and medical diagnostic technology and, more specifically, to medical devices configured with needle electrodes.

### Description of the Related Art

In minimally invasive medical procedures, a healthcare professional typically inserts a medical device into the patient's body and positions an instrument head of the medical device near tissue of a particular tissue type, such as a tumor. Some medical devices include a needle that penetrates the tissue. The needle can be fixed or retractable, where mechanical and/or electrical actuators are implemented to extend and retract the needle, and typically is used to administer various forms of treatment. For example, the needle can be used to extract a sample from a tumor for visual inspection or biopsy, deliver a diagnostic agent, such as a visual and/or radiolabeling dye, and/or deliver a therapeutic agent, such as a therapeutic drug or energy.

One drawback of many conventional medical devices, where a needle forms part of the instrument head, is the difficulty in determining, during treatment, whether the tissue type at the location of the needle tip matches an expected tissue type at that location. For example, a healthcare professional can visually inspect an image captured by a camera to determine the tissue type near the tip of a needle and administer treatment after visually confirming that the tissue appears to be a tumor. However, tissue types can vary in appearance, and different tissue types can have similar appearances. Accordingly, visual inspections can be inaccurate. As another example, tissue of one type may be located within tissue of another type, such as a tumor embedded within healthy tissue. A healthcare professional can use a needle to penetrate the healthy tissue and reach the targeted tissue. However, capturing an image of the target tissue at the location of the needle tip prior to, and during, this type of treatment can be difficult, if not impossible. Accordingly, verifying by visual inspection that the tissue type at the location of the needle tip is that of a tumor also can be quite difficult, if not impossible.

In view of the above drawbacks, some medical devices, where a needle forms part of the instrument head, include components that enable the tissue contacting the needle to be evaluated and a tissue type to be determined. However, many techniques for determining tissue type are inaccurate and, accordingly, are insufficient for confirming that the tissue type at the location of a needle tip matches an expected tissue type. For example, a medical scan can indicate a targeted tissue type at a given location, and triangulation and ultrasound imaging can confirm that a needle is positioned at the location where the targeted tissue type is expected to exist. However, these techniques typically require calibrating the relevant positioning system with respect to both the instrument head and a mapping of the patient's body via a medical scan. Errors introduced in the calibration process can produce errors when determining whether the instrument head, including the needle, is positioned correctly at the location of the targeted tissue type. Also, any physiological changes within the patient, such as the size, shape, or location of a tumor, between the time when a medical scan is conducted and a time point when the medical procedure begins can potentially change the tissue type at the target location. Thus, positioning an instrument head based on a medical scan can result in applying a needle to healthy tissue instead of the target tissue.

As the foregoing illustrates, what is needed in the art are more effective techniques for determining the tissue type at the location of a needle tip for medical devices, where a needle forms part of the instrument head.

### SUMMARY

Embodiments are disclosed for medical devices. In various embodiments, the medical device includes an instrument head that includes a needle and two or more electrodes disposed on the needle; an impedance bridge coupled to the two or more electrodes; and a processor coupled to the impedance bridge.

Embodiments are disclosed for deploying a medical device. In various embodiments, a method includes recording, at one or more frequencies, one or more impedance measurements, wherein each impedance measurement is associated with two or more electrodes disposed on the needle of an instrument head of the medical device; comparing the one or more impedance measurements to one or more characteristic impedances associated with one or more tissue types; and determining, based on the one or more impedance measurements and the one or more characteristic impedances, one or more tissue types at the location associated with the needle.

At least one technical advantage of the disclosed design relative to the prior art is that the disclosed medical device is able to automatically determine, during operation, the tissue type of tissue at a location associated with a needle included in an instrument head of the medical device. For example, the disclosed medical device can determine whether the tissue type of tissue at the location associated with the needle matches an expected tissue type at that location prior to or during a procedure that involves the needle, such as extracting a tissue sample and/or delivering a therapeutic drug or energy to the tissue. In this manner, the disclosed medical device can ensure that the needle is applied to a correctly targeted tissue type, such as a tumor, rather than some other tissue type, such as healthy tissue. Also, the disclosed medical device can apply the needle to targeted tissue types more accurately than is possible with conventional medical devices. Consequently, the disclosed medical device can be used to perform various procedures with respect to targeted tissue types, such as and without limitation, delivering therapeutic drugs or energy or extracting tissue samples, more accurately and reliably than what can be achieved using conventional medical devices. These technical advantages provide one or more technological advancements over prior art designs and approaches.

### BRIEF DESCRIPTION OF THE DRAWINGS

FIG. 1 illustrates a medical device, according to various embodiments;

FIG. 2 is a more detailed illustration of the instrument head of FIG. 1, according to various embodiments;

FIG. 3 is a more detailed illustration of the instrument head of FIG. 1, according to other various embodiments;

FIG. 4A is a more detailed illustration of the instrument head of FIG. 1, according to other various embodiments;

FIG. 4B is a close-up illustration of the needle tip of FIG. 4A, according to various embodiments;

FIG. 5 is a more detailed illustration of the external electrical components of FIG. 1, according to various embodiments;

FIG. 6 is a more detailed illustration of the medical device of FIG. 1, according to various embodiments; and

FIG. 7 is a flow diagram of method steps for determining one or more tissue types at a location associated with a needle of a medical device, according to various embodiments.

### DETAILED DESCRIPTION

In the following description, numerous specific details are set forth to provide a more thorough understanding of the various embodiments. However, in the range of embodiments of the concepts includes some embodiments omitting one or more of these specific details.

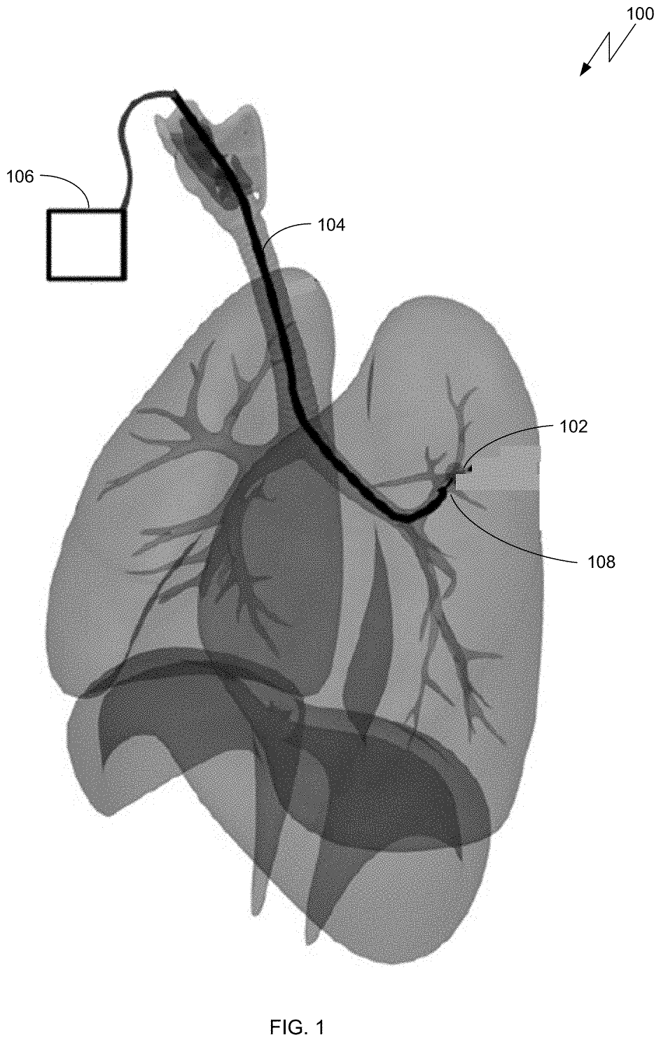

FIG. 1 illustrates a medical device 100, according to various embodiments. As shown, the medical device 100 includes, without limitation, an instrument head 108, wires 104, and external electrical components 106. The instrument head 108 is positioned at a location 102 (e.g., a location of a tumor). While not shown, the instrument head 108 includes a needle. While not shown, in some embodiments, the instrument head 108 also includes a medical device tool, such as and without limitation, a camera, a fiber optic light source, a therapeutic drug delivery tool that delivers a therapeutic drug to the location 102, an energy delivery tool that delivers energy to the location 102, or a tissue sample extraction tool that extracts a tissue sample from the location 102 for further evaluation. The external electrical components 106 generate current at various frequencies. The wires 104 conduct the current between the external electrical components 106 and the instrument head 108. The external electrical components 106 include a processor that couples to two or more electrodes disposed on the needle. The processor of the external electrical components 106 measures the impedance of current conducted through tissue between at least two of the two or more electrodes. As described in greater detail below, the medical device 100 determines, based on the impedance measurements, one or more tissue types at the location associated with the needle. For example and without limitation, based on the impedance measurements, the tissue type can indicate whether tissue at the location associated with the needle is a tumor tissue type or a non-tumor tissue type.

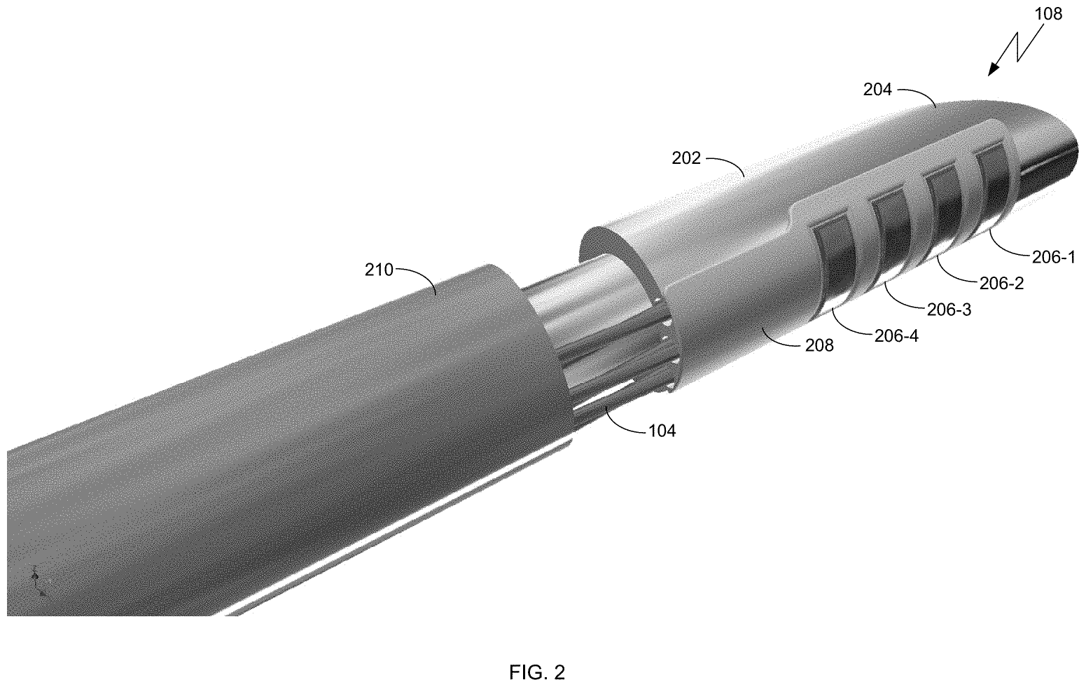

FIG. 2 is a more detailed illustration of the instrument head 108 of FIG. 1, according to various embodiments. As shown, the instrument head 108 includes, without limitation, a needle 202 and a sheath 210, and wires 104. As shown, the needle 202 includes a needle tip 204, electrodes 206-1 through 206-4, and electrically insulating material 208.

In various embodiments, the electrically insulating material 208 is located between adjacent pairs of electrodes 206. As shown, the electrically insulating material 208 includes a set of carve-outs, and each of the two or more electrodes is located within one of the carve-outs of the electrically insulating material 208. In various embodiments and without limitation, the electrodes 206 are disposed on top of the electrically insulating material 208. The electrically insulating material 208 can reduce contact and short circuits between adjacent electrodes 206, which could reduce the accuracy of the impedance measurements.

As shown, each of the two or more electrodes 206-1 through 206-4 is located at a respective location along the length of the needle 202. For example and without limitation, the first electrode 206-1 is located at a first location near the needle tip 204, and the second electrode 206-2 is located at a second location that is further from the needle tip 204. When the instrument head 108 is positioned at a location 102 in the patient's body, the needle 202 penetrates tissue at the location 102. The external electrical components 106 can record, at one or more frequencies, one or more impedance measurements, wherein each impedance measurement is associated with two or more of the electrodes 206. For example and without limitation, the medical device can record a first impedance measurement of tissue between or contacting the first electrode 206-1 and the second electrode 206-2 and a second impedance measurement of tissue between or contacting the third electrode 206-3 and the fourth electrode 206-4. Based on the first and second impedance measurements, the medical device can determine a first tissue type between the first electrode 206-1 and the second electrode 206-2 and a second tissue type between the third electrode 206-3 and the fourth electrode 206-4. For example and without limitation, the first tissue type based on the first impedance measurement can indicate that the tissue near the needle tip 204 is a tumor, and the second tissue type based on the second impedance measurement can indicate that the tissue further from the needle tip 204 is not a tumor. Based on the tissue types at the location associated with the needle, the external electrical components 106 can determine that the needle tip 204 of the needle 202 has penetrated healthy tissue to the depth of the embedded tumor. However, if the first impedance measurement and the second impedance measurement are the same or similar to one another, or to characteristic impedance measurements of non-tumor tissue, the external electrical components 106 can determine that the needle tip 204 of the needle 202 has not yet penetrated healthy tissue to the depth of the embedded tumor, or that a tumor is not present at the location associated with the needle 202.

While not shown, in some embodiments, one or more of the two or more electrodes 206 is located on a first side of the needle 202, and at least another one or more of the two or more electrodes is located on a second side of the needle 202. For example and without limitation, the first electrode 206-1 and the second electrode 206-2 could be located on a left side of the needle 202, and the third electrode 206-3 and the fourth electrode 206-4 could be located on a right side of the needle 202. The external electrical components 106 can selectably couple to the first electrode 206-1 and the second electrode 206-2 to record a first impedance measurement. Based on the first impedance measurement, the external electrical components 106 can determine a tissue type on the left side of the needle 202. The external electrical components 106 can selectably couple to the third electrode 206-3 and the fourth electrode 206-4 to record a second impedance measurement. Based on the second impedance measurement, the external electrical components 106 can determine a tissue type on the right side of the needle 202. Based on the first and second impedance measurements, the external electrical components 106 can compare the tissue types on different sides of the needle 202. For example and without limitation, the external electrical components 106 can determine whether the needle 202 is located within a tumor, within healthy tissue, or between a tumor and healthy tissue.

While not shown, in some embodiments, one or more of the two or more electrodes 206 is located on a first side of the needle 202, and the needle 202 selectably rotates with respect to a length axis of the needle 202. For example and without limitation, the medical device 100 can include an actuator (e.g. and without limitation, a stepper motor or servo motor), and the actuator is coupled to the instrument head 108 and that, when actuated, rotates the needle 202. For example, and without limitation, when the instrument head 108 is positioned at a location 102 in the patient's body, the external electrical components 106 can actuate the actuator to rotate the needle 202 to a first rotational position in which the electrodes 206 are located on a left side of the needle 202. The external electrical components 106 can record a first impedance measurement to determine a tissue type of tissue associated with the left side of the needle 202. The external electrical components 106 can actuate the actuator again to rotate the needle 202 to a second rotational position in which the electrodes 206 are located on a right side of the needle 202. The external electrical components 106 can record a first impedance measurement to determine a tissue type of tissue associated with the right side of the needle 202. The external electrical components 106 can compare the tissue types on the left side and the right side of the needle 202. For example and without limitation, the external electrical components 106 can determine whether the needle 202 is located within a tumor, within healthy tissue, or between a tumor and healthy tissue.

As shown, the electrodes 206-1 through 206-4 are disposed on the needle. The wires 104 electrically couple the electrodes 206-1 through 206-4 to the external electrical components 106. The sheath 210 physically protects the wires 104, and, in some embodiments, shields the wires from electromagnetic interference.

While not shown, in some embodiments, the needle 202 selectably extends by an adjustable amount relative to the sheath 210. In various embodiments, the needle 202 can move between a first position that is fully retracted into the sheath 210 (e.g., during deployment or movement of the instrument head 108) and a second position that is fully extended from the sheath 210 (e.g., when the instrument head 108 is positioned at the target location 102). For example and without limitation, the needle 202 can be coupled to a wire that extends through the sheath 210 to the external electrical components 106. In some embodiments, an actuator included in the external electrical components 106 exerts pressure on the cable to extend the needle 202 with respect to the sheath 210. In some embodiments, an actuator included in the external electrical components 106 exerts tension on the cable to retract the needle 202 out of the sheath 210. In some embodiments, the external electrical components 106 automatically move the needle 202 based on the one or more tissue types at the location associated with the needle. For example and without limitation, when the external electrical components 106 determine that the tissue type at the location associated with the instrument head 108 matches an expected tissue type at the target location 102, the external electrical components 106 can actuate the actuator to extend the needle 202 with respect to the sheath 210 and penetrate the tissue. In some embodiments, the adjustable amount of the needle 202 extending relative to the sheath 210 includes three or more positions in which the needle 202 is fully exposed, partially exposed, or fully retracted into the sheath 210.

While not shown, in some embodiments, the sheath 210 selectably extends by an adjustable amount relative to the needle 202. In various embodiments, the sheath 210 can move between a first position that covers the needle 202 (e.g., during deployment or movement of the instrument head 108) and a second position that retracts the sheath 210 with respect to the needle 202 (e.g., when the instrument head 108 is positioned at the target location 102). For example and without limitation, the sheath 210 can be coupled to a wire that extends to the external electrical components 106. In some embodiments, an actuator included in the external electrical components 106 can exert pressure on the cable to extend the sheath 210 to cover the needle 202. In some embodiments, an actuator included in the external electrical components 106 can exert tension on the cable to retract the sheath 210 and expose the needle 202. In some embodiments, the external electrical components 106 automatically move the sheath 210 based on the one or more tissue types at the location associated with the needle. For example and without limitation, when the external electrical components 106 determine that the tissue type at the location associated with the instrument head 108 matches an expected tissue type at the target location 102, the external electrical components 106 can actuate the actuator to retract the sheath 210 and expose the needle 202 to penetrate the tissue. In some embodiments, the adjustable amount of the sheath 210 extending relative to the needle 202 includes three or more positions in which the needle 202 is fully exposed, partially exposed, or fully retracted into the sheath 210.

While not shown, in various embodiments, the sheath 210 includes one or more carve-outs, and the sheath 210 selectably rotates to expose or cover the electrodes 206. For example and without limitation, the external electrical components 106 can include an actuator (e.g. and without limitation, a stepper motor or servo motor), and the actuator is coupled to the instrument head 108 and that, when actuated, rotates the needle 202. The sheath 210 can selectably rotate between a first rotational position in which the one or more carve-outs exposes a first electrode 206 of the two or more electrodes 206, and a second rotational position in which the sheath 210 covers the first electrode 206. For example, and without limitation, when the instrument head 108 is being moved, the external electrical components 106 can actuate the actuator to rotate the sheath 210 to a first rotational position in which the sheath 210 covers the first electrode 206-1 and the carve-outs do not expose the first electrode 206-1. Covering the first electrode 206-1 can protect the first electrode during movement of the instrument head 108. When the instrument head is positioned at a location in the patient's body, the external electrical components 106 can actuate the actuator to rotate the sheath 210 to a second rotational position in which one or more of the carve-outs in the sheath 210 exposes the first electrode 206-1. Exposing the first electrode 206-1 can enable the external electrical components 106 to measure impedance measurements of tissue between the first electrode 206-1 and another one of the electrodes 206. In some embodiments, different rotational positions of the sheath 210 expose different respective subsets of the electrodes 206 and cover the remaining electrodes 206 disposed on the needle 202. In some embodiments, a first rotational position of the sheath 210 covers all of the electrodes 206 disposed on the needle 202, and a second rotational position of the sheath 210 exposes all of the electrodes 206 through the carve-outs of the sheath 210.

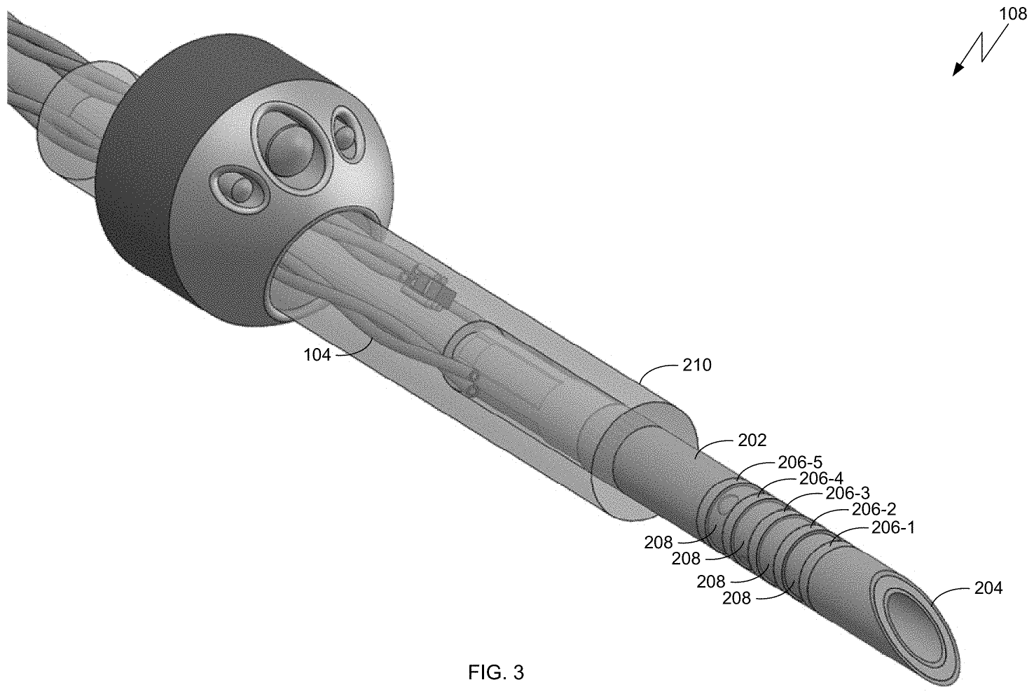

FIG. 3 is a more detailed illustration of the instrument head of FIG. 1, according to other various embodiments. As shown, the instrument head 108 includes, without limitation, a needle 202, wires 104, and a sheath 210. As shown, the needle 202 includes a needle tip 204 and a set of electrodes 206-1 to 206-5, and electrically insulating material 208.

As shown, each of the electrodes 206 encircles the needle 202 at a respective location along the length of the needle 202. For example and without limitation, the first electrode 206-1 encircles the needle 202 at a first location near the needle tip 204, and the second electrode 206-2 encircles the needle 202 at a second location that is further from the needle tip 204. When the instrument head 108 is positioned at a location 102 in the patient's body, the needle 202 penetrates tissue at the location 102. The external electrical components 106 can record, at one or more frequencies, one or more impedance measurements, wherein each impedance measurement is associated with two or more of the electrodes 206. For example and without limitation, the medical device can record a first impedance measurement of tissue between or contacting the first electrode 206-1 and the second electrode 206-2 and a second impedance measurement of tissue between or contacting the third electrode 206-3 and the fourth electrode 206-4. Based on the first and second impedance measurements, the medical device can determine a first tissue type between the first electrode 206-1 and the second electrode 206-2 and a second tissue type between the third electrode 206-3 and the fourth electrode 206-4. For example and without limitation, the first tissue type based on the first impedance measurement can indicate that the tissue near the needle tip 204 is a tumor, and the second tissue type based on the second impedance measurement can indicate that the tissue further from the needle tip 204 is not a tumor. Based on the tissue types at the location associated with the needle, the external electrical components 106 can determine that the needle tip 204 of the needle 202 has penetrated healthy tissue to the depth of the embedded tumor. However, if the first impedance measurement and the second impedance measurement are the same or similar to one another, or to characteristic impedance measurements of non-tumor tissue, the external electrical components 106 can determine that the needle tip 204 of the needle 202 has not yet penetrated healthy tissue to the depth of the embedded tumor, or that a tumor is not present at the location associated with the needle 202.

As shown, the electrodes 206 are disposed on top of a layer of electrically insulating material 208. The electrically insulating material 208 is also located between adjacent pairs of electrodes 206. As shown, the electrically insulating material 208 encircles the needle 202 between each pair of adjacent electrodes 206. The electrically insulating material 208 can reduce contact and short circuits between adjacent electrodes 206, which could reduce the accuracy of the impedance measurements.

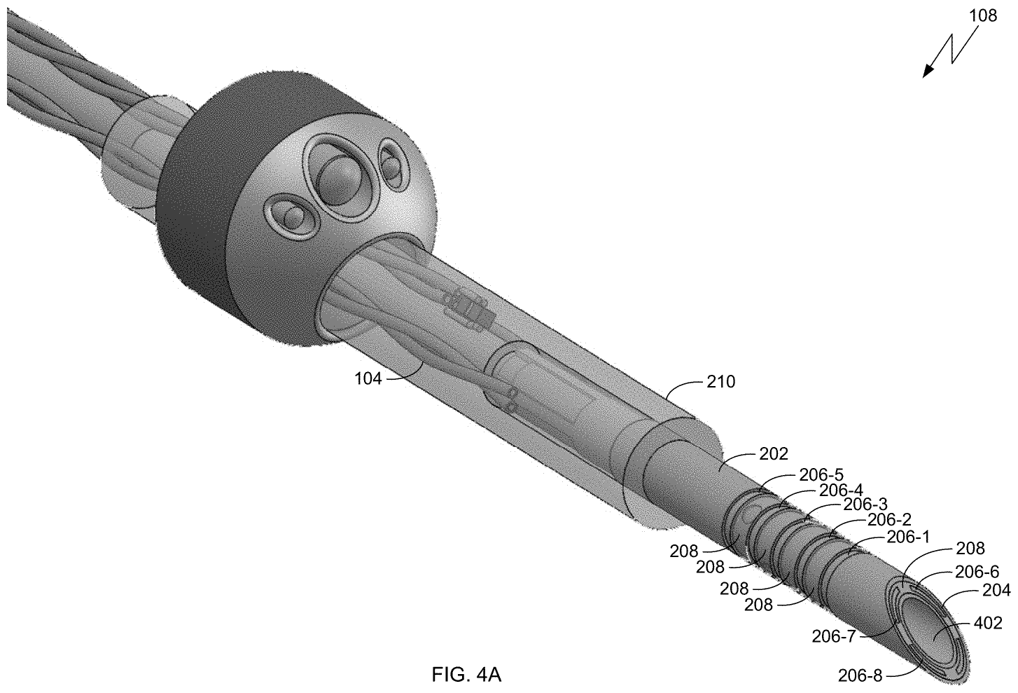

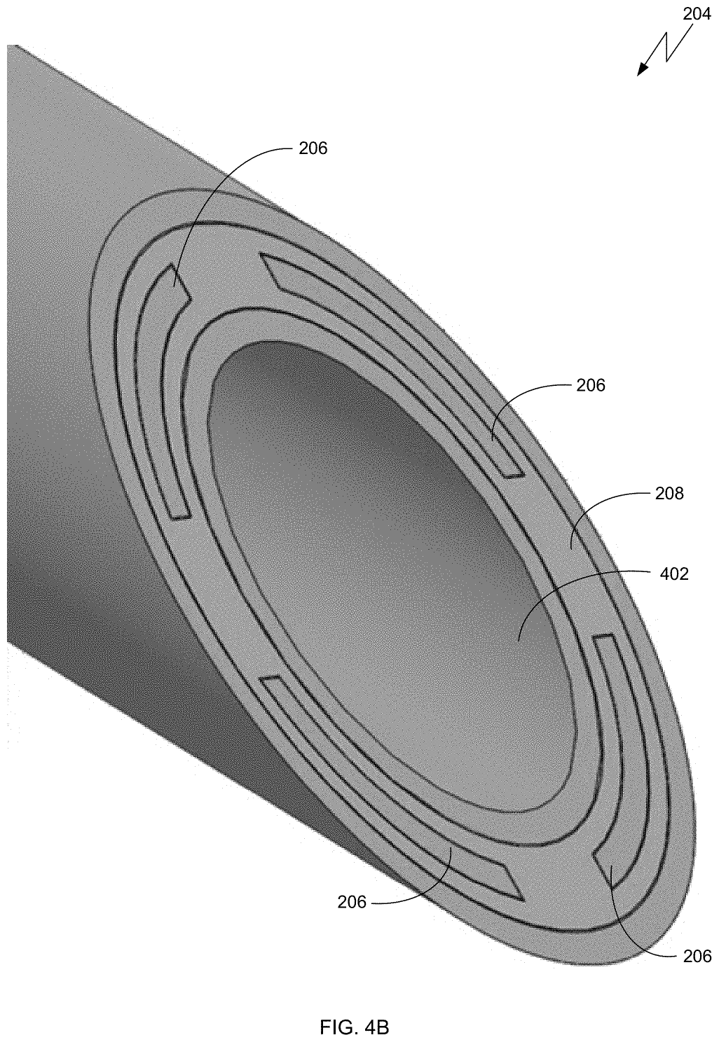

FIG. 4A is a more detailed illustration of the instrument head of FIG. 1, according to other various embodiments. As shown, the instrument head 108 includes, without limitation, a needle 202 including a cannula terminating in an aperture 402, wires 104, and a sheath 210. As shown, the needle 202 includes a needle tip 204 and a set of electrodes 206-1 to 206-8, and electrically insulating material 208.

As shown, each of the electrodes 206 encircles the needle 202 at a respective location along the length of the needle 202. For example and without limitation, the first electrode 206-1 encircles the needle 202 at a first location near the needle tip 204, and the second electrode 206-2 encircles the needle 202 at a second location that is further from the needle tip 204. In addition, as shown, electrodes 206-6 to 206-8 are disposed on the needle tip surrounding an aperture 402 of the cannula. When the instrument head 108 is positioned at a location 102 in the patient's body, the needle 202 penetrates tissue at the location 102. The external electrical components 106 can record, at one or more frequencies, one or more impedance measurements, wherein each impedance measurement is associated with two or more of the electrodes 206. For example and without limitation, the medical device can record a first impedance measurement of tissue between or contacting the sixth electrode 206-6 and the seventh electrode 206-7 and a second impedance measurement of tissue between or contacting the first electrode 206-1 and the second electrode 206-2. Based on the first and second impedance measurements, the medical device can determine a first tissue type between the sixth electrode 206-6 and the seventh electrode 206-7 and a second tissue type between the first electrode 206-1 and the second electrode 206-2. For example and without limitation, the first tissue type based on the first impedance measurement can indicate that the tissue contacting the needle tip 204 is a tumor, and the second tissue type based on the second impedance measurement can indicate that the tissue further from the needle tip 204 is not a tumor. Based on the one or more tissue types at the location associated with the needle, the external electrical components 106 can determine that the needle tip 204 of the needle 202 has penetrated healthy tissue to the depth of the embedded tumor. However, if the first impedance measurement and the second impedance measurement are the same or similar to one another, or to characteristic impedance measurements of non-tumor tissue, the external electrical components 106 can determine that the needle tip 204 of the needle 202 has not yet penetrated healthy tissue to the depth of the embedded tumor, or that a tumor is not present at the location associated with the needle 202.

FIG. 4B is a close-up illustration of the needle tip of FIG. 4A, according to various embodiments. As shown, the needle tip 204 includes an aperture 402 of a cannula, one or more electrodes 206, and electrically insulating material 208.

As shown, the electrodes 206 are disposed on the needle tip 204 at locations surrounding the aperture 402 of the cannula. For example and without limitation, in various embodiments in which the needle 202 is included in a therapeutic drug delivery tool, the cannula can convey a therapeutic drug through the aperture 402 and into tissue that is penetrated by the needle tip 204. For example and without limitation, in various embodiments in which the needle 202 is included in an energy delivery tool, the cannula can convey energy through the aperture 402 and into tissue that is penetrated by the needle tip 204. For example and without limitation, in various embodiments in which the needle 202 is included in a tissue sample extraction tool, the cannula can receive and store a tissue sample of tissue that is penetrated by the needle tip 204.

When the instrument head 108 is positioned at a location 102 in the patient's body, the needle 202 penetrates tissue at the location 102. The external electrical components 106 can record, at one or more frequencies, one or more impedance measurements, wherein each impedance measurement is associated with two or more of the electrodes 206. For example and without limitation, the medical device can record a first impedance measurement of tissue between or contacting the sixth electrode 206-6 and the seventh electrode 206-7 and a second impedance measurement of tissue between or contacting the first electrode 206-1 and the second electrode 206-2. Based on the first and second impedance measurements, the medical device can determine a first tissue type between the sixth electrode 206-6 and the seventh electrode 206-7 and a second tissue type between the first electrode 206-1 and the second electrode 206-2. For example and without limitation, the first tissue type based on the first impedance measurement can indicate that the tissue contacting the needle tip 204 is a tumor, and the second tissue type based on the second impedance measurement can indicate that the tissue further from the needle tip 204 is not a tumor. Based on the one or more tissue types at the location associated with the needle, the external electrical components 106 can determine that the needle tip 204 of the needle 202 has penetrated healthy tissue to the depth of the embedded tumor. However, if the first impedance measurement and the second impedance measurement are the same or similar to one another, or to characteristic impedance measurements of non-tumor tissue, the external electrical components 106 can determine that the needle tip 204 of the needle 202 has not yet penetrated healthy tissue to the depth of the embedded tumor, or that a tumor is not present at the location associated with the needle 202.

While not shown, in various embodiments, one or more of the two or more electrodes is disposed on an interior surface of a cannula of the needle 202. For example and without limitation, in various embodiments in which the needle 202 is included in a tissue extraction tool, the cannula of the needle 202 can receive and store a tissue sample of tissue that is penetrated by the needle tip 204. The external electrical components 106 can record one or more impedance measurements of the tissue sample between the electrodes 206 disposed on the interior surface of the cannula. Based on the one or more impedance measurements, the external electrical components 106 can determine a tissue type of the tissue sample.

While not shown, in some embodiments, the needle includes a first cannula and a second cannula that resides within the first cannula. For example and without limitation, the first cannula can include two or more electrodes are disposed on an interior surface of the first cannula, and the second cannula can perform one or more operations, such as delivering a therapeutic drug or energy or extracting a tissue sample. The external electrical components 106 can record one or more impedance measurements of the tissue sample between the electrodes 206 disposed on the interior surface of the first cannula and can determine a tissue type of tissue contacting the interior surface of the first cannula. Based on the one or more impedance measurements and the one or more tissue types at the location associated with the needle, the external electrical components 106 can determine whether or not to perform the one or more operations involving the second cannula. For example and without limitation, if the one or more tissue types at the location associated with the needle is a tumor, the external electrical components 106 can actuate a drug delivery tool to dispense a therapeutic drug from the second cannula. However, if the one or more tissue types at the location associated with the needle include a healthy tissue type, the external electrical components 106 can refrain from actuating the drug delivery tool to avoid dispensing the therapeutic drug to the healthy tissue. As another example and without limitation, the first cannula can perform one or more operations, such as delivering a therapeutic drug or energy or extracting a tissue sample, and the second cannula can include two or more electrodes are disposed on an interior surface of the second cannula. The external electrical components 106 can record one or more impedance measurements of the tissue sample between the electrodes 206 disposed on the interior surface of the second cannula and can determine a tissue type of tissue contacting the interior surface of the second cannula. Based on the one or more impedance measurements and the one or more tissue types at the location associated with the needle, the external electrical components 106 can determine whether or not to perform the one or more operations involving the first cannula. For example and without limitation, if the one or more tissue types at the location associated with the needle include a tumor tissue type, the external electrical components 106 can actuate a drug delivery tool to dispense a therapeutic drug from the first cannula. However, if the one or more tissue types at the location associated with the needle include a healthy tissue type, the external electrical components 106 can refrain from actuating the drug delivery tool to avoid dispensing the therapeutic drug to the healthy tissue.

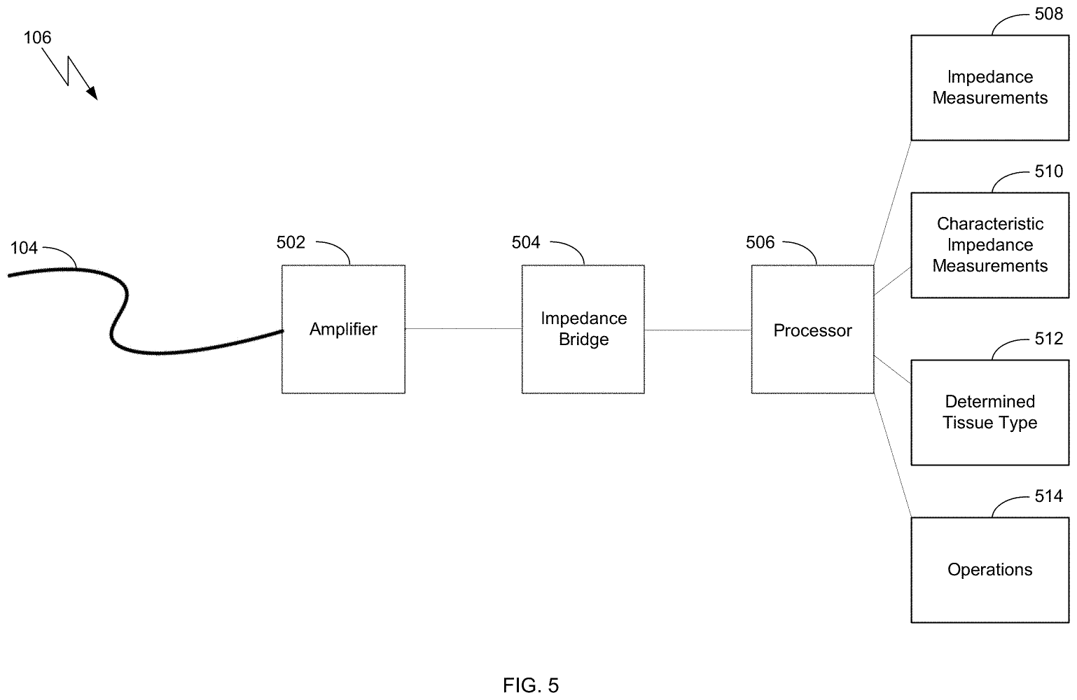

FIG. 5 is a more detailed illustration of the external electrical components of FIG. 1, according to various embodiments. As shown, the external electrical components 106 include wires 104, an amplifier 502, an impedance bridge 504, and a processor 506. The wires 104 conduct current at various frequencies between two or more electrodes 206 and the external electrical components 106. In various embodiments, the amplifier 502 is an analog interface amplifier that amplifies a supplied voltage and/or a return voltage while the wires 104 conduct current at various frequencies between the impedance bridge 504 and the two or more electrodes 206. In various embodiments, the impedance bridge 504 is an impedance load that the processor 506 measures to determine an impedance of a circuit including the impedance bridge 504, the amplifier 502, and the two or more electrodes 206. The processor 506 generates frequencies for a current that the wires 104 conduct between the impedance bridge 504 and the selected two or more electrodes 206.

While the wires 104 conduct current at various frequencies, the processor 506 records one or more impedance measurements 508 of the circuit including the at least two electrodes 206. The processor 506 compares the one or more impedance measurements 508 with characteristic tissue types 512 of respective one or more tissue types. Based on the one or more impedance measurements 508 and the characteristic tissue types 512, the processor 506 determines one or more tissue types 512 of tissue at the location associated with the needle 202. For example and without limitation, based on the one or more impedance measurements 508 and the characteristic tissue types 512, the processor 506 can determine which tissue type is associated with characteristic impedance measurements 510 that are closest to the impedance measurements of the portion of tissue between at least two of the two or more electrodes 206. In various embodiments, the processor 506 can determine a Cole relaxation frequency of the portion of tissue based on the impedance measurements 508, and can compare the Cole relaxation frequency to one or more characteristic Cole relaxation frequencies of one or more tissue types. The Cole relaxation frequency corresponds to a frequency associated with a greatest impedance measurement 508 included in the one or more impedance measurements 508. In various embodiments, the Cole relaxation frequency is a frequency of a maximum normalized impedance measurement of the portion of tissue between at least two of the two or more electrodes 206. For example and without limitation, based on a Cole relaxation frequency below a threshold frequency (e.g., 10⁵ Hz), the processor 506 can determine that the portion of tissue between at least two of the two or more electrodes 206 is a non-tumor tissue type. Similarly, for example and without limitation, based on a Cole relaxation frequency above the threshold frequency, the processor 506 can determine that the portion of tissue between at least two of the two or more electrodes 206 is a tumor tissue type. In various embodiments, the processor 506 determines one or more tissue types at the location associated with the needle 202.

In some embodiments, the processor 506 determines two or more tissue types of tissue at the location associated with the needle based on impedance measurements respectively recorded by different electrode pairs of the two or more electrodes 206. For example (without limitation), the needle 202 can include a first electrode pair disposed at a first location along the length of the needle 202 and a second electrode pair disposed at a second location along the length of the needle 202. While the needle 202 is inserted into tissue, the processor 506 can determine tissue types at the first location and the second location based on the respective impedance measurements associated with the first electrode pair and the second electrode pair. The one or more tissue types at the location associated with the needle can indicate whether the needle 202 has been inserted into tissue far enough to reach a location of a targeted tissue type within the tissue.

In various embodiments in which the instrument head 108 includes a sheath 210 and a needle 202 that selectably extends by an adjustable amount relative to the sheath 210, the processor 506 can extend the needle to a first amount relative to the sheath 210. For example and without limitation, when the instrument head 108 is moving, the processor 506 can actuate an actuator to retract the needle 202 fully into the sheath 210. When the processor 506 determines that the tissue type 512 at the location associated with the needle 202 matches one or more tissue types at a target location 102, the processor 506 can actuate the actuator to extend the needle 202 with respect to the sheath. Selectably extending the needle 202 can protect the needle 202 while the instrument head 108 is moving and can prevent the needle 202 from penetrating tissue at a location other than the target location 102.

In various embodiments in which the instrument head 108 includes a needle 202 and a sheath 210 that selectably extends by an adjustable amount relative to the needle 202, the processor 506 can extend the sheath 210 to a first amount relative to the needle 202. For example and without limitation, when the instrument head 108 is moving, the processor 506 can actuate an actuator to extend the sheath 210 to cover the needle 202. When the processor 506 determines that the tissue type 512 at the location associated with the needle 202 matches one or more tissue types at a target location 102, the processor 506 can actuate the actuator to retract the sheath 210 with respect to the sheath and to expose the needle 202. Selectably extending the sheath 210 can protect the needle 202 while the instrument head 108 is moving and can prevent the needle 202 from penetrating tissue at a location other than the target location 102.

In various embodiments in which the instrument head 108 includes a medical device tool, the processor 506 performs one or more operations 514 to control the medical device tool based on the one or more tissue types 512 at the location associated with the needle. For example and without limitation, in various embodiments in which the instrument head 108 includes a camera, the processor 506 can perform operations 514 that include activating the camera to capture an image of the tissue at the location 102 associated with the needle 202. For example and without limitation, in various embodiments in which the instrument head 108 includes a light source, the processor 506 can perform operations 514 that include activating the light source to illuminate the tissue at the location 102 associated with the needle 202. For example and without limitation, in various embodiments in which the needle 202 is included in a therapeutic drug delivery tool, the processor 506 can perform operations 514 that include activating the therapeutic drug delivery tool to deliver one or more therapeutic drugs to the tissue at the location 102 associated with the needle 202. For example and without limitation, in various embodiments in which the needle 202 is included in an energy delivery tool, the processor 506 can perform operations 514 that include activating the energy delivery tool to deliver energy to the tissue at the location 102 associated with the needle 202. For example and without limitation, in various embodiments in which the needle 202 is included in a tissue sample extraction tool, the processor 506 can perform operations 514 that include activating the tissue sample extraction tool to extract a tissue sample from the tissue at the location 102 associated with the needle 202.

In various embodiments in which the needle 202 is included in a medical device tool, the processor 506 determines one or more tissue types at a location associated with the needle 202 at a time point, where the time point is either prior or subsequent to a medical procedure. For example and without limitation, in various embodiments in which the needle 202 is included in a therapeutic drug delivery tool, the processor 506 can record a first one or more impedance measurements 508 to determine the tissue type 512 at the location associated with the needle 202 at a first time point that is before delivery of the therapeutic drug. The processor 506 can also record a second one or more impedance measurements 508 to determine the tissue type 512 at the location associated with the needle 202 at a second time point that is after delivery of the therapeutic drug. Determining the tissue types 512 at time points before and after the delivery of the therapeutic drug can indicate changes to the tissue due to the delivered therapeutic drug. For example and without limitation, in various embodiments in which the needle 202 is included in an energy delivery tool, the processor 506 can record a first one or more impedance measurements 508 to determine the tissue type 512 at the location associated with the needle 202 at a first time point that is before delivery of the energy. The processor 506 can also record a second one or more impedance measurements 508 to determine the tissue type 512 at the location associated with the needle 202 at a second time point that is after delivery of the energy. Determining the tissue types 512 at time points before and after the delivery of the therapeutic drug can indicate changes to the tissue due to the delivered energy.

In various embodiments, the processor 506 presents the one or more tissue types at the location 102 associated with the needle 202. For example and without limitation, the processor 506 can display the one or more tissue types 512 at the location associated with the needle using a visual output (e.g., a light-emitting diode, a liquid crystal display, or the like). For example and without limitation, where the target location 102 is a tumor that is embedded in non-tumor tissue, the displayed tissue type 512 can indicate whether the needle 202 has penetrated the non-tumor tissue to the depth of the tumor. Presenting the indication can inform a user of the medical device 100 that the one or more tissue types at the location 102 associated with the needle the instrument head 108 matches the expected tissue type at a target location. Further, in various embodiments, the processor 506 performs the one or more operations 514 to control a medical device tool based on presenting the tissue type 512 and receiving a signal to activate the medical device tool.

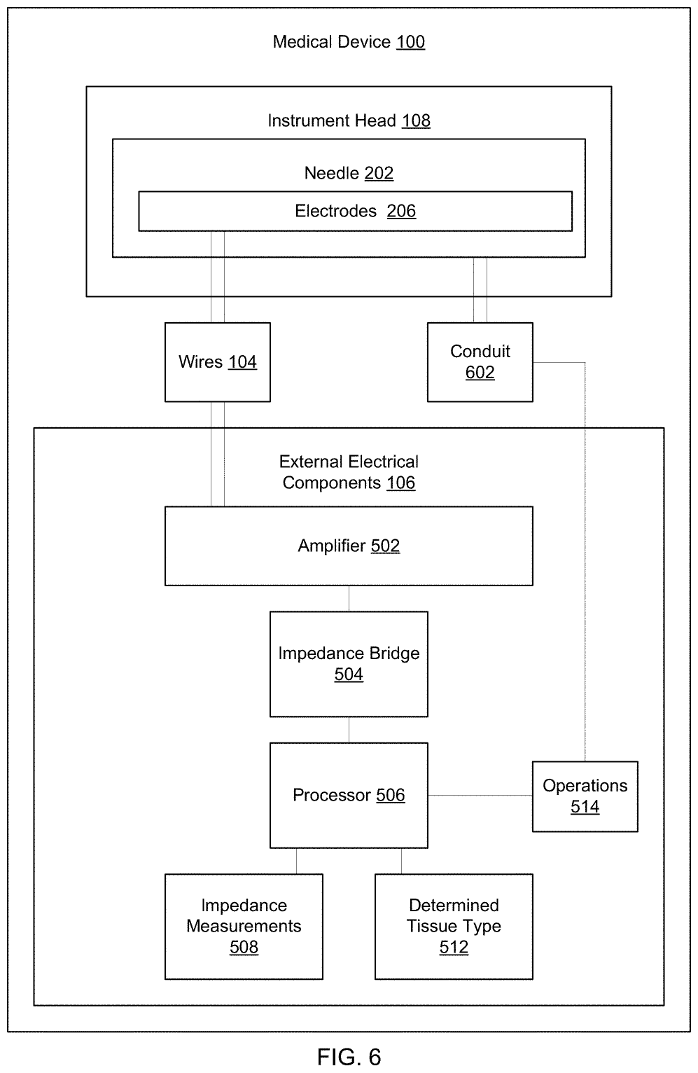

FIG. 6 is a more detailed illustration of the medical device 100 of FIG. 1, according to various embodiments. As shown, the medical device 100 includes an instrument head 108 and external electrical components 106. As shown, the instrument head 108 includes a needle 202 and two or more electrodes 206 that are disposed on the needle 202. The two or more electrodes 206 are coupled to the external electrical components 106 by wires 104. In various embodiments, without limitation, each of the two or more electrodes 206 is coupled to the external electrical components 106 by one wire 104 or by respective wires of a plurality of wires 104. In some embodiments, the instrument head 108 includes a medical device tool, such as and without limitation, a therapeutic drug delivery tool, an energy delivery tool, or a tissue sample extraction tool. In various embodiments, the instrument head 108 includes, without limitation, two or more medical device tools, which can be of one kind or of different kinds.

As shown, the external electrical components 106 include an amplifier 502, an impedance bridge 504, and a processor 506. The amplifier 502 amplifies a supplied voltage and/or a return voltage while the wires 104 conduct current at various frequencies between the impedance bridge 504 and the two or more electrodes 206. The impedance bridge 504 is an impedance load that the processor 506 measures to determine an impedance of a circuit including the impedance bridge 504, the amplifier 502, the wires 104, and the two or more electrodes 206. The processor 506 records, at various frequencies, one or more impedance measurements 508. The processor 506 compares the two or more impedance measurements 508 with characteristic impedance measurements 510 of respective one or more tissue types. Based on the one or more impedance measurements 508 with characteristic tissue types 512, the processor 506 determines one or more tissue types 512 at the location 102 associated with the needle 202. In various embodiments and without limitation, the processor 506 determines the tissue type 512 indicated by the respective impedance measurements 508 based on a Cole relaxation frequency of a portion of tissue contacting the two or more electrodes 206. In various embodiments and without limitation, the processor 506 determines the tissue type 512 as areas of tumor tissue types and/or non-tumor tissue types. In various embodiments and without limitation, based on the one or more tissue types 512 at the location associated with the needle, the processor 506 determines that the tissue type at the location 102 associated with the needle 202 matches the expected tissue type at a target location, which indicates or confirms that the instrument head 108 is positioned at the target location 102. For example and without limitation, if the target location 102 is a tumor, the processor 506 can determine whether the needle 202 is positioned at a target location 102 by determining that the one or more tissue types 512 at the location associated with the needle include a tumor tissue type.

As shown, the processor 506 is coupled to a conduit 602 that is coupled to the needle 202. Based on the one or more tissue types 512 at the location associated with the needle, the processor 506 performs one or more operations 514 to control the needle 202. In various embodiments and without limitation, the needle 202 is included in a therapeutic drug delivery tool, and the processor 1906 performs an operation 514 of causing the needle 202 to deliver one or more therapeutic drugs to tissue at the location 102 associated with the needle 202. For example and without limitation, the processor 506 can cause one or more therapeutic drugs through one or more drug delivery conduits to and through the needle 202. In various embodiments and without limitation, the needle 202 is included in an energy delivery tool, and the processor 506 performs an operation 514 of causing the conduit 602 and the needle 202 to deliver energy to tissue at the location 102 associated with the needle 202. For example and without limitation, the processor 506 can cause current to be conducted through wires in the conduit 602 to and through the needle 202. In various embodiments and without limitation, the needle 202 is included in a tissue sample extraction tool, and the processor 506 performs an operation 514 of causing the needle 202 to extract a tissue sample from tissue at the location 102 associated with the needle 202. For example and without limitation, the external electrical components 106 can include an actuator coupled to the needle 202 by wires in the conduit 602, and the processor 506 can activate the actuator to cause the needle 202 to extract the tissue sample.

In various embodiments, the medical device 100 reports the one or more tissue types 512 at the location associated with the needle to a user of the medical device 100. For example and without limitation, the medical device 100 can display the one or more tissue types 512 at the location associated with the needle using a visual output (e.g., a liquid crystal display (LCD), a light-emitting diode (LED) display to present a visual indication of the one or more tissue types 512 at the location associated with the needle, such as a light, symbol, text, graphic, or the like). In various embodiments and without limitation, the processor 506 can include, in the displayed tissue type 512, an indication that the one or more tissue types 512 at the location associated with the needle match one or more expected tissue types at a target location (e.g., using a visual output, an audio output, or the like).

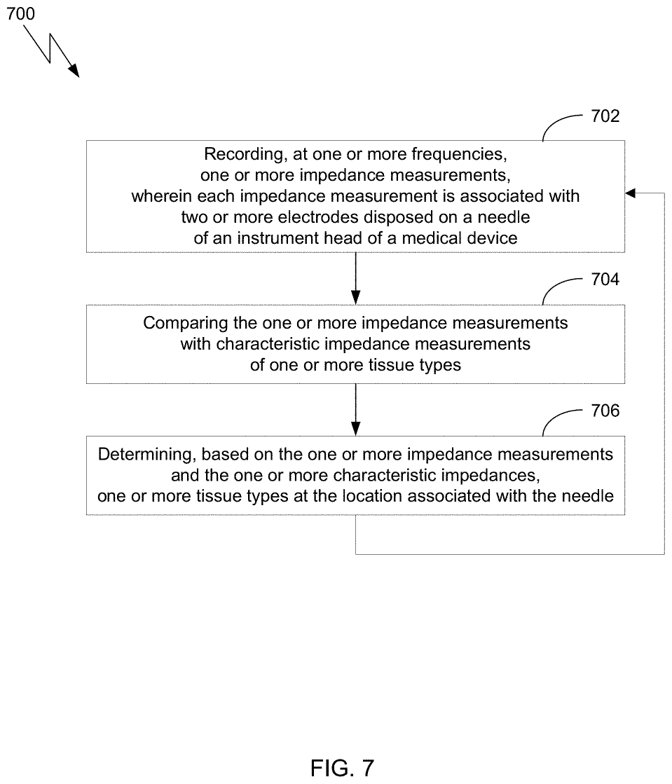

FIG. 7 is a flow diagram of method steps for controlling the medical device 100 of FIG. 1, according to various embodiments. Although the method steps are described in conjunction with the systems of FIGS. 1-6, persons skilled in the art will understand that any system configured to perform the method steps, in any order, falls within the scope of the present invention.

As shown, a method 700 begins at step 702, where a processor 506 records, at one or more frequencies, one or more impedance measurements 508, wherein each impedance measurement 508 is associated with two or more electrodes 206 disposed on a needle 202 included in an instrument head 108 of the medical device 100. In various embodiments and without limitation, the processor 506 determines a Cole relaxation frequency of tissue between at least two of the two or more electrodes disposed on the needle (e.g., without limitation, as a frequency of a maximum normalized impedance measurement of the tissue between at least two of the two or more electrodes).

At step 704, the processor compares the one or more impedance measurements with characteristic impedance measurements of respective one or more tissue types. For example and without limitation, the processor can compare the impedance measurements with a first set of one or more characteristic impedance measurements of a non-tumor tissue type and a second set of one or more characteristic impedance measurements of a tumor tissue type.

At step 706, the processor determines, based on the two or more impedance measurements, one or more tissue types at the location associated with the needle. In various embodiments and without limitation, the processor determines the tissue type that classifies the tissue as one of a tumor tissue type or a non-tumor tissue type. In various embodiments and without limitation, the processor determines whether the tissue type at the location associated with the location matches an expected tissue type at a target location. The method can return to step 702 to record additional impedance measurements and to determine a second or updated tissue type.

In sum, the disclosed medical device measures the impedance of tissue in a location where a needle of an instrument head of a medical device is positioned. The medical device determines the tissue type based on impedance measurements associated with two or more electrodes located on the needle. The disclosed approach advantageously results in the medical device determining the tissue types of tissue associated with the location associated with the needle (e.g., without limitation, on various sides of the instrument head).

At least one technical advantage of the disclosed medical device relative to the prior art is that the disclosed medical device is able to determine one or more tissue types of tissue associated with a location associated with the needle of an instrument head of the medical device prior to performing an operation associated with the needle during the operation. For example (without limitation), the disclosed medical device can determine whether the tissue type of the tissue associated with the location associated with the needle matches an expected tissue type at a given target location prior to or during administering a treatment using the needle, such as extracting a tissue sample and/or delivering a therapeutic drug or energy. In this manner, the disclosed medical device can ensure that the needle is applied to a selected tissue type, such as a tumor, rather than some other tissue type, such as healthy tissue. Also, the disclosed medical device can apply the needle at target locations more accurately than is possible with conventional medical devices. Consequently, the disclosed medical device can be used to perform various procedures based on the needle, such as and without limitation, delivering therapeutic drugs or energy or extracting tissue samples, at specific locations more accurately and reliably than what can be achieved using conventional medical devices. These technical advantages provide one or more technological advancements over prior art approaches.

1. In some embodiments, a medical device comprises an instrument head that includes a needle and two or more electrodes disposed on the needle; an impedance bridge coupled to the two or more electrodes; and a processor coupled to the impedance bridge.

2. The medical device of clause 1, wherein each of the two or more electrodes is located at a respective location along a length of the needle.

3. The medical device of clauses 1 or 2, wherein at least one of the two or more electrodes is located on a first side of the needle, and at least another one of the two or more electrodes is located on a second side of the needle.

4. The medical device of any of clauses 1-3, wherein at least one of the two or more electrodes encircles the needle.

5. The medical device of any of clauses 1-4, wherein at least one of the two or more electrodes is located on a tip of the needle.

6. The medical device of any of clauses 1-5, wherein the needle includes an electrically insulating material that is located between at least two of the two or more electrodes.

7. The medical device of any of clauses 1-6, wherein the needle includes an electrically insulating material, and each of the two or more electrodes is disposed on top of the electrically insulating material.

8. The medical device of any of clauses 1-7, wherein the needle includes an electrically insulating material, and each of the two or more electrodes is disposed within a different carve-out within the electrically insulating material.

9. The medical device of any of clauses 1-8, wherein one or more of the two or more electrodes is disposed on an interior surface of a cannula of the needle.

10. The medical device of any of clauses 1-9, wherein the needle includes a first cannula and a second cannula that resides within the first cannula, and the two or more electrodes are disposed on either the first cannula or the second cannula.

11. The medical device of any of clauses 1-10, wherein the instrument head includes a sheath, and the needle selectably extends by an adjustable amount relative to the sheath.

12. The medical device of any of clauses 1-11, wherein the instrument head includes a sheath that selectably extends by an adjustable amount relative to the needle.

13. The medical device of any of clauses 1-12, wherein the instrument head includes a sheath that has one or more carve-outs, and the sheath selectably rotates between a first rotational position, in which the one or more carve-outs exposes at least one of the two or more electrodes, and a second rotational position, in which the sheath covers the at least one of the two or more electrodes.

14. In some embodiments, a method for determining one or more tissue types at a location associated with a needle of a medical device comprises recording, at one or more frequencies, one or more impedance measurements, wherein each impedance measurement is associated with two or more electrodes disposed on the needle of an instrument head of the medical device; comparing the one or more impedance measurements to one or more characteristic impedances associated with one or more tissue types; and determining, based on the one or more impedance measurements and the one or more characteristic impedances, one or more tissue types at the location associated with the needle.

15. The method of clause 14, wherein the one or more impedance measurements include a first impedance measurement associated with an electrode pair included in the two or more electrodes.

16. The method of clauses 14 or 15, wherein the needle selectably extends by an adjustable amount relative to a sheath included in the instrument head, and the method further comprises, based on the one or more tissue types at the location associated with the needle, extending the needle to a first amount relative to the sheath.

17. The method of any of clauses 14-16, further comprising, based on the one or more tissue types at the location associated with the needle, performing one or more operations to control a medical device tool included in the instrument head.

18. The method of any of clauses 14-17, further comprising receiving a tissue sample at the location associated with the needle, wherein the recorded impedance measurements include one or more impedance measurements of the tissue sample.

19. The method of any of clauses 14-18, wherein at least one of the one or more impedance measurements is associated with at least two electrodes located on a side of the needle.

20. The method of any of clauses 14-19, wherein recording at least one of the one or more impedance measurements occurs prior or subsequent to a medical procedure.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A medical device, comprising:
an instrument head that includes:
a needle, and
two or more electrodes disposed on the needle;
an impedance bridge coupled to the two or more electrodes; and
a processor coupled to the impedance bridge.

2. The medical device of claim 1, wherein each of the two or more electrodes is located at a respective location along a length of the needle.

3. The medical device of claim 1, wherein at least one of the two or more electrodes is located on a first side of the needle, and at least another one of the two or more electrodes is located on a second side of the needle.

4. The medical device of claim 1, wherein at least one of the two or more electrodes encircles the needle.

5. The medical device of claim 1, wherein at least one of the two or more electrodes is located on a tip of the needle.

6. The medical device of claim 1, wherein the needle includes an electrically insulating material that is located between at least two of the two or more electrodes.

7. The medical device of claim 1, wherein the needle includes an electrically insulating material, and each of the two or more electrodes is disposed on top of the electrically insulating material.

8. The medical device of claim 1, wherein the needle includes an electrically insulating material, and each of the two or more electrodes is disposed within a different carve-out within the electrically insulating material.

9. The medical device of claim 1, wherein one or more of the two or more electrodes is disposed on an interior surface of a cannula of the needle.

10. The medical device of claim 1, wherein the needle includes a first cannula and a second cannula that resides within the first cannula, and the two or more electrodes are disposed on either the first cannula or the second cannula.

11. The medical device of claim 1, wherein the instrument head includes a sheath, and the needle selectably extends by an adjustable amount relative to the sheath.

12. The medical device of claim 1, wherein the instrument head includes a sheath that selectably extends by an adjustable amount relative to the needle.

13. The medical device of claim 1, wherein the instrument head includes a sheath that has one or more carve-outs, and the sheath selectably rotates between a first rotational position, in which the one or more carve-outs exposes at least one of the two or more electrodes, and a second rotational position, in which the sheath covers the at least one of the two or more electrodes.

14. A method for determining one or more tissue types at a location associated with a needle of a medical device, the method comprising:
recording, at one or more frequencies, one or more impedance measurements, wherein each impedance measurement is associated with two or more electrodes disposed on the needle of an instrument head of the medical device;
comparing the one or more impedance measurements to one or more characteristic impedances associated with one or more tissue types; and
determining, based on the one or more impedance measurements and the one or more characteristic impedances, one or more tissue types at the location associated with the needle.

15. The method of claim 14, wherein the one or more impedance measurements include a first impedance measurement associated with an electrode pair included in the two or more electrodes.

16. The method of claim 14, wherein the needle selectably extends by an adjustable amount relative to a sheath included in the instrument head, and the method further comprises, based on the one or more tissue types at the location associated with the needle, extending the needle to a first amount relative to the sheath.

17. The method of claim 14, further comprising, based on the one or more tissue types at the location associated with the needle, performing one or more operations to control a medical device tool included in the instrument head.

18. The method of claim 14, further comprising receiving a tissue sample at the location associated with the needle, wherein the recorded impedance measurements include one or more impedance measurements of the tissue sample.

19. The method of claim 14, wherein at least one of the one or more impedance measurements is associated with at least two electrodes located on a side of the needle.

20. The method of claim 14, wherein recording at least one of the one or more impedance measurements occurs prior or subsequent to a medical procedure.
